(19) United States
(12) Patent Application Publication
(10) Pub. No.: US 2025/0120580 A1
(43) Pub. Date: Apr. 17, 2025

(54) DEVICES, SYSTEMS, AND RELATED METHODS FOR DELIVERING AGENTS TO BODY LUMENS

(71) Applicant: Boston Scientific Scimed, Inc., Maple Grove, MN (US)

(72) Inventors: Siavash Sam PARKHIDEH, Marlborough, MA (US); Barry WEITZNER, Acton, MA (US); Samuel GOSHEN, Brookline, MA (US); Lauren Sfakis LYDECKER, Millbury, MA (US)

(73) Assignee: Boston Scientific Scimed, Inc., Maple Grove, MN (US)

(21) Appl. No.: 18/913,470

(22) Filed: Oct. 11, 2024

Related U.S. Application Data
(60) Provisional application No. 63/589,717, filed on Oct. 12, 2023.

(51) Int. Cl. A61B 1/05; A61B 1/00; A61B 1/313

(52) U.S. Cl. CPC A61B 1/05; A61B 1/00011; A61B 1/3137

(57) ABSTRACT

Systems and methods for a medical device are disclosed. For example, the medical device may be configured to release at least one agent from a housing upon interacting with a substance. The medical device may include a capsule wall defining the at least one housing, and a sensor configured to detect the substance. The housing may be configured to contain the at least one agent.

### CROSS-REFERENCE TO RELATED APPLICATIONS

This application claims the benefit of priority under 35 U.S.C. § 119 from U.S. Provisional Application No. 63/589,717, filed Oct. 12, 2023, which is incorporated by reference herein in its entirety.

### TECHNICAL FIELD

Various aspects of this disclosure relate generally to medical devices, systems, and related methods thereof. More specifically, embodiments of the disclosure relate to medical devices and systems for treating, locating, and/or imaging a luminal lesion, for example, a bleed within a wall of a small intestine.

### BACKGROUND

Gastrointestinal bleeding is a serious issue that can lead to death if not rapidly and properly addressed. Small intestinal bleeding can result from arteriovenous malformations (AVMs), tumors, polyps, Crohn's disease, and/or ulcers, and is most common in individuals over age 50. Typical treatment includes hospitalization to stabilize blood pressure and heart rate, as well as providing IV fluids and blood transfusions. Endoscopic access or surgery may be needed to locate and stop the bleeding.

### SUMMARY

Each of the aspects disclosed herein may include one or more of the features described in connection with any of the other disclosed aspects.

Aspects of the disclosure relate to a medical device that may include a capsule wall defining at least one housing and a sensor configured to detect a substance. The housing may be configured to contain at least one agent. The medical device may be configured to release the at least one agent from the housing upon interacting with the substance.

Any of the devices disclosed herein may include any of the following features, in any combination. The at least one agent may include a hemostatic agent or a dye. The sensor may include a camera, wherein the substance is blood, and wherein the camera is configured to detect blood. The camera may be configured to detect a fluorescent signature of blood. The capsule wall may include an emulsion polymer configured to degrade in a basic solution. The housing may comprise more than one sub-housing. The sensor may include an electronic component configured to communicate the detection of the substance to a user device. The substance may include one or more of an acidic environment, a basic environment, or an activating substance.

The medical device may include a release mechanism. The release mechanism may include a degradable material configured to degrade upon contact with the substance. The release mechanism may be a pressurized release mechanism. The activating substance may be administered intravenously or orally to a patient. The activating substance may be at least one of an activating molecule, an enzyme, or a fluorophore. The capsule wall may include at least one aperture in a wall. The at least one aperture may be configured to transition from a closed state to an open state upon detection of the substance. Each aperture of the at least one aperture may include a degradable material configured to degrade upon detection of the substance, such that the at least one aperture is transitioned from a closed state to an open state. At least a portion of the capsule may include a degradable material.

In another example, the medical device may include a capsule wall defining at least one housing and a sensor configured to detect blood. The housing may be configured to contain at least one agent. The sensor may include an electronic component configured to communicate with a user device.

Any of the devices disclosed herein may include any of the following features, alone or in any combination. The capsule may be configured to deliver the at least one agent based on an instruction from the user device. The sensor may be a camera configured to detect a fluorescent signature of blood.

A method may include administering a capsule to a patient, and upon interaction with a substance in a body lumen of the patient, automatically releasing at least one agent from the capsule. The capsule may contain the at least one agent in an interior of the capsule.

Any exemplary methods disclosed herein may include any of the following steps, alone or in any combination. The capsule may include a wall. The method may further include automatically releasing the agent by degradation of the wall upon interaction with the substance. The capsule may include a sensor configured to detect the substance. The method may further include automatically releasing the agent when the sensor detects the substance.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction further into a body of a subject, and the term “proximal” refers to an opposite direction (e.g., a direction toward a subject's mouth). The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1A illustrates an exemplary medical device in a closed state, according to one or more embodiments.

FIG. 1B illustrates the exemplary medical device of FIG. 1A in an open state.

FIG. 2A illustrates an alternative medical device in a closed state, according to one or more embodiments.

FIG. 2B illustrates a cross-sectional view of the medical device of FIG. 2A.

FIG. 3 depicts a medical system, according to one or more embodiments.

FIG. 4 illustrates a related method of using an exemplary medical device, such as the medical devices of FIGS. 1-2B.

FIGS. 5A-5D illustrate aspects of the exemplary method of FIG. 4.

FIG. 6 depicts a simplified functional block diagram of a computer, according to one or more embodiments.

### DETAILED DESCRIPTION

Embodiments of this disclosure relate to medical devices and systems including structures configured to detect, treat, and/or manage luminal lesions, e.g., bleeds in the small bowel. Gastrointestinal (GI) bleeding is a serious issue that can lead to death if not rapidly and properly addressed. Small intestinal bleeding can result from, for example, arteriovenous malformations (AVMs), tumors, polyps, Crohn's disease, and ulcers, and is most common in individuals over age 50. Typical treatment includes hospitalization to stabilize blood pressure and heart rate, as well as providing IV fluids and blood transfusions. Endoscopic access or surgery may be needed to locate and stop the bleeding. However, while many regions of the GI tract are accessible endoscopically, much of the small intestine cannot be accessed, making specialized techniques and/or surgery, which may be invasive, necessary to address bleeding.

Although treatment of small intestinal bleeding is referenced herein, it will be appreciated that the disclosed devices and methods may be utilized in the course of other treatments (for example, during a variety of medical procedures that involve locating, treating, diagnosing, and/or managing issues relating lumens of the body).

A medical device of this disclosure may be a capsule configured to release an agent to a body lumen of a subject. In one example, the capsule may include an electronic sensor that is configured to sense the presence of blood. Upon the sensor detecting blood, the capsule may be configured to deploy an agent, such as an agent, to treat the bleeding. In another example, the capsule may include a shell configured to degrade or automatically break apart in the jejunum and ileum to deliver an agent contained within the capsule. For example, the shell may include a material that is non-permeable in acidic conditions and breaks apart in basic conditions. In another example, the capsule may carry a marking agent that marks a treatment area (e.g., a treatment area with blood) for later medical treatment (e.g., surgical treatment).

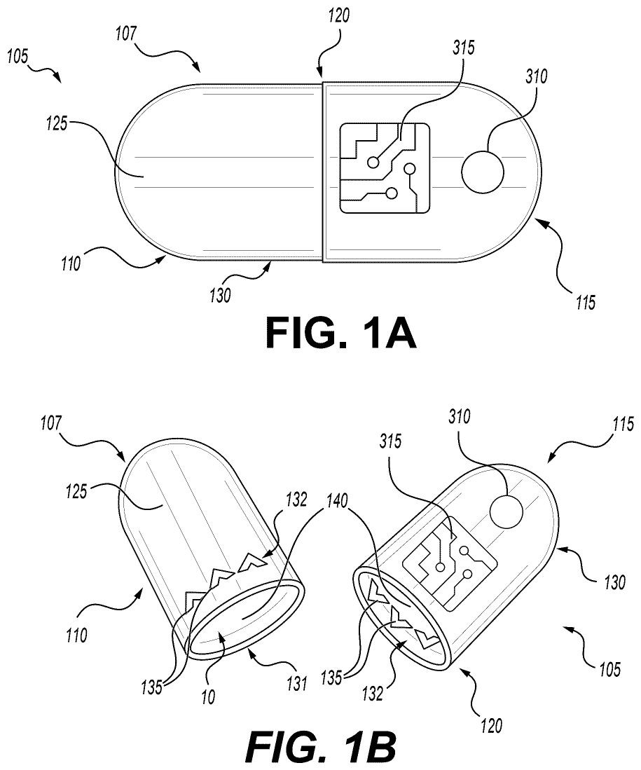

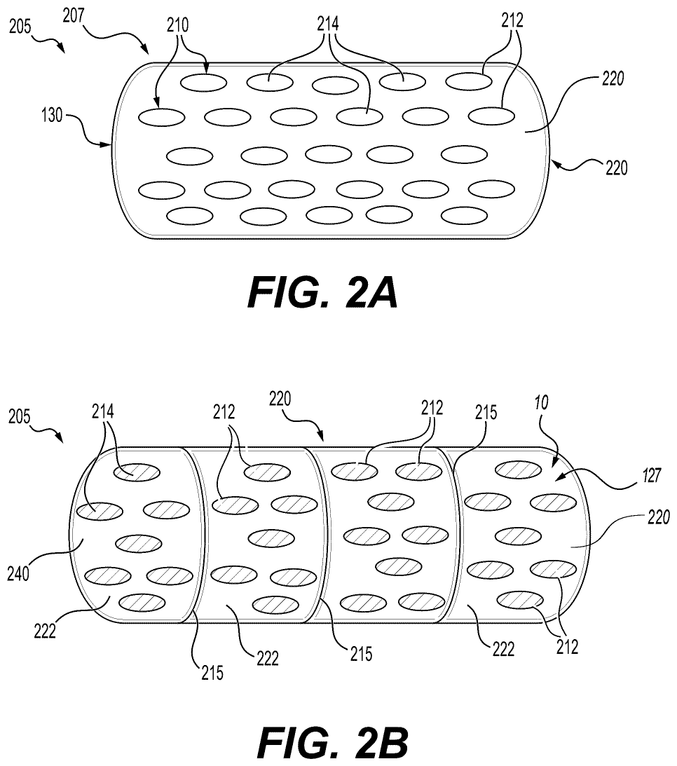

FIGS. 1A and 1B illustrate a first embodiment of a medical device 105 that may be configured to deploy to a treatment site (e.g., a lesion such as an injury) to detect a substance and/or release a hemostatic or other agent. FIGS. 2A and 2B depict a second embodiment of a medical device 205 that may similarly be deployed to a treatment site, as discussed in further detail below. Medical devices 105, 205 may be capsules or may be similar to capsules. Medical devices 105, 205 may improve access to various body regions, e.g., areas of the small intestine, for locating and/or treating bleeds. Where not otherwise specified, it will be appreciated that medical devices 105, 205 may have any of the same properties. Medical devices 105, 205 may be configured to house and/or release a hemostatic or other agent, e.g., in response to detecting or interacting with a substance.

Examples of agents that may be disposed within or otherwise deployed by medical devices 105, 205 may include at least one of a powder, a fluid, a foam, a clay (e.g., kaolin clay, bentonite clay, etc.), a patch, a molecule or compound (e.g., a pharmaceutical compound, a coagulant, calcium, fibrin, etc.), a biologic, a mechanical tamponade (e.g., gel foam, starch, chitosan, cellulose, etc.), and/or a cautery agent. In alternatives, medical devices 105, 205 may have housed therein other agent(s), such as a marking agent, e.g., a biocompatible dye (e.g., methylene blue, patent blue, brilliant blue, endoscopic tattoo, etc.), etc. Delivery of a biocompatible dye by devices 105, 205 may enable location/marking of the lesion, e.g., via X-ray, computerized topography (CT) scan, magnetic resonance imaging (MRI) scan, etc. The hemostatic or other agent may be attracted to exposed cells of the lesion and/or other substances at or around the site of the lesion, such that at least a portion of the hemostatic or other agent becomes concentrated at the injury site.

As mentioned above and discussed in further detail below, in some examples, medical device 105, 205 may be configured to deploy its payload (e.g., a hemostatic or marking agent) in response to properties of a lesion site and/or nearby anatomy. In some examples, medical device 105, 205 may be configured to deliver its payload in the presence of blood. In addition or in the alternative, medical device 105, 205 may be configured to deliver its payload in response to presence of a substance, such as an activating molecule, an acidic environment, a basic environment, an enzyme, a fluorophore, etc.

In some embodiments, medical device 105 (FIGS. 1A and 1B) may include a capsule 107, a camera 310 or other imaging sensor, and/or a sensor 315. As used herein, the term “camera” includes a sensor configured to detect any type of optical input. Similarly, medical device 205 (FIGS. 2A and 2B) may include a capsule 207. Capsule 107, 207 may have any suitable configuration, e.g., a two-piece capsule (as depicted for, e.g., capsule 107), a one-piece capsule (as depicted for, e.g., capsule 207), etc.

Capsule 107, 207 may be comprised of any suitable material or combination of materials. In some examples, capsule 107, 207 may be disposable. In other examples, capsule 107, 207 (or portions thereof) may be reusable. As discussed in more detail below, capsule 107, 207 (and/or portions thereof) may include materials or components configured to degrade, dissolve, disintegrate, signal, or otherwise react in the presence of a substance at or near a lesion site (e.g., in the presence of blood, an activating substance, a basic environment, an acidic environment, etc.). Capsule 107, 207 (and/or portions thereof) may be configured to maintain stability in the presence of other bodily fluids.

Capsule 107 (FIGS. 1A and 1B) may include an interior housing 140 (FIG. 1B) defined by a capsule wall 125. Interior housing 140 may be configured to house at least one agent 10. In some examples, interior housing 140 may be a compartment within a portion/subset of capsule 107. In other examples, interior housing 140 may extend through an entirety of an interior capsule 107. Interior housing 140 may have any suitable size or shape. A size, shape, or other properties of interior housing 140 may be chosen based on a payload (e.g., agent 10) of interior housing 140.

Capsule 207 may similarly include an interior housing 240 (FIG. 2B) defined by a capsule wall 220, having any of the properties of capsule wall 125. Interior housing 240 may have any of the features of interior housing 140. In some embodiments, as depicted for capsule 207 in FIG. 2B, interior housing 240 may include at least one interior wall 215. Capsule 107 may also include such interior walls, although not shown in FIGS. 1A and 1B. Interior wall 215 may be comprised of any suitable material, such as materials configured to break down (e.g., gelatin, cellulose, etc.) or materials configured to remain intact through an entirety of the body lumen (e.g., polymer or metal). At least one interior sub-housing 222 may be defined by at least one interior wall 215 and capsule wall 220 of capsule 207. Capsule 107, 207 may include any suitable number of sub-housings 222. For example, capsule 107, 207 may include one, two, three, four, etc. interior sub-housings 222. Each of the at least one interior sub-housing 222 may house at least one payload (e.g., hemostatic or other agent). For example, a first interior sub-housing may house a first agent, a second interior sub-housing may house a second agent, a third interior sub-housing may house a third agent, a fourth interior sub-housing may house a marking agent, and a fifth interior sub-housing may house another treatment agent, etc.

As shown in FIGS. 1A and 1B, capsule 107 may include a capsule body 110 and/or a capsule cap 115 connected at a connection point 120. In some embodiments, connection point 120 may be pressurized. Connection point 120 may be a conventional connection point, e.g., interlocking rings, snap-fit, etc., or spring-loaded, and/or may include a release mechanism, as discussed in further detail below. Alternatively, capsule 107 may include a single outer capsule wall 125, with an opening formed therein, which may be covered by a cover having a connection point similar to connection point 120, and having any of the properties thereof.

In some embodiments, connection point 120 may include a release mechanism (hereinafter “interaction point release mechanism”) 132, e.g., a release mechanism including at least one interaction point 135. Interaction point release mechanism 132 may be configured to enable release of a payload (e.g., a hemostatic agent or marking agent) from capsule 107. Interaction point release mechanism 132 may release and/or cause to release the at least one agent based on detection of a substance within a body lumen or a property of the body lumen (e.g., via camera 310 or sensor 315, receipt of a communication (e.g., from a user device), etc.), as described in further detail below. Upon activation of interaction point release mechanism 132, the payload may diffuse from an area of high concentration (e.g., an interior of the capsule) to an area of lower concentration (e.g., into the body lumen of the patient).

Interaction point release mechanism 132 may be spring-loaded and/or may include a material configured to activate (e.g., degrade) in a presence of certain substances. A spring-loaded interaction point release mechanism 132 may be controllable by a user, as described in further detail herein. A degradable material of interaction point release mechanism 132 may be in any suitable configuration. For example, as depicted in FIG. 1B, at least one interaction point 135 may be located on an exterior rim of capsule body 110 and/or an interior rim of capsule cap 115, such that the at least one interaction point 135 interact when the exterior rim of capsule body 110 and the interior rim of capsule cap 115 are connected. In some examples, interaction point release mechanism 132 may be configured disintegrate, degrade, etc. in the presence of certain substances (according to any of the mechanisms discussed herein for capsule wall 125), thereby releasing capsule body 110 from capsule cap 115. Interaction point release mechanism 132 may be pressurized, e.g., via the connection between capsule body 110 and capsule cap 115. For example, if medical device 105 is pressurized, the pressurization may aid in the separation of capsule body 110 from capsule cap 115, thereby releasing the at least one agent.

In some embodiments, medical devices 105 or 205 may include other release mechanism configurations. For example, capsule wall 125, 220 may include a release mechanism (hereinafter “capsule wall release mechanism”) in that capsule wall 125, 220 may be composed of a material configured to degrade, disintegrate, etc. in the presence of a substance (e.g., blood), such that, as medical devices 105, 205 interact with (e.g., encounters) blood in the body lumen of a patient (e.g., due to the presence of a bleed in the luminal wall), capsule wall 125, 220 may degrade, enabling the release of the at least one agent. In some example, capsule wall 125, 220 may include numerous portions having the same or different materials. For example, a portion of capsule 107, 207 may include a capsule wall 125, 220 that is comprised of, for example, biocompatible metal and/or polymers that may pass through an entirety of a body lumen and be expelled via a natural orifice of the body (e.g., the anus). For example, a portion of capsule 107 housing camera 310 and/or sensor 315 may be formed from a material that is intended to pass through the body lumen in such a manner. Another portion of capsule 107, 207 (e.g., a portion of capsule 107, 207 in that forms at least a portion of interior housing 140, 240) may be configured to degrade within the body lumen.

In some embodiments, all or portions of capsule wall 125, 220 may be composed of a material configured to degrade in the presence of an activating substance. For example, the activating substance may be intravenously and/or orally administered to the patient and may be present at the site of injury, e.g., at a bleed in the patient's small intestine. Medical device 105, 205 may interact with the activating substance in the body lumen of a patient (e.g., due to the presence of a bleeding injury in the luminal wall). The interaction may cause all or a portion of capsule wall 125, 220 to degrade, enabling the release of the at least one agent. The material of capsule wall 125, 220 (or a portion thereof) may be configured to degrade in the presence of the activating substance while not degrading in other portions of the body lumen.

In some embodiments, portions or an entirety of capsule wall 125, 220 may be composed of a material configured to degrade in an acidic solution, such that, as medical device 105, 205 passes through an acidic environment of the stomach and/or duodenum, capsule wall 125, 220 (or portions thereof) may degrade, enabling the release of a payload of medical device 105.

In additional or alternative embodiments, portions or an entirety of capsule wall 125, 220 may be covered by or composed of a material configured to degrade in a basic solution (e.g., an enteric coating 130, labeled in FIGS. 1A and 1B), such that as medical device 105, 205 interacts with a basic environment of the jejunum and/or ileum, enteric coating 130 may degrade, enabling the release of the at least one agent. In such examples, capsule wall 125, 220 (or a portion thereof) may remain intact as medical device 105, 205 passes through the stomach and duodenum, due to an acidic environment of the stomach and duodenum.

Enteric coating 130 may be configured to prevent and/or reduce degradation of capsule 107, 207 in acidic environments, e.g., in stomach acid. Enteric coating 130 may be comprised of any suitable material or combination of materials, such as emulsion polymers, including poly(2-dimethylamino)ethylmethacrylate), Chitosan, P(HEMA), Methyl acrylate-methacrylic acid copolymers, Cellulose acetate phthalate (CAP), Cellulose acetate succinate, Hydroxypropyl methyl cellulose phthalate, Hydroxypropyl methyl cellulose acetate succinate (hypromellose acetate succinate), Polyvinyl acetate phthalate (PVAP), Methyl methacrylate-methacrylic acid copolymers, Shellac, Cellulose acetate trimellitate, Sodium alginate, Zein, etc.

In some embodiments, capsule wall 125, 220 may be composed of more than one material, e.g., a first material, a second material, etc. The first material may include enteric coating 130 or another coating, which may be an outermost coating of capsule 107, 207. The second material may be an inner or innermost layer of capsule 107, 207 (hereinafter “interior material”) 131 (labeled in FIG. 1B) configured to degrade in the presence of a substance in the body lumen. The first material (e.g., enteric coating 130) may be configured to be external/outward relative to the second material. For example, capsule wall 125, 220 may be composed of an outermost layer of enteric coating 130 that degrades as medical device 105 interacts with the acidic solution of the stomach, exposing interior material 131 to the basic solution of the jejunum and/or ileum. As medical device 105 interacts with the basic solution, the second, interior material 131 of capsule wall 125, 220 may degrade, enabling the release of the payload (e.g., agent or marking agent).

Further release mechanism configurations of capsules 107, 207 may include a plurality of apertures, in some examples. Such a release mechanism is depicted for capsule 207 in FIG. 2A but may be utilized with any of the capsules described herein. As shown in FIG. 2A, capsule 207 may include a plurality of aperture release mechanisms 210. Aperture release mechanisms 210 may enable sustained release of the agent. Each aperture release mechanism 210 may include an aperture 212 and/or a material configured to degrade in the presence of a substance (hereinafter “degradable material”) 214. For example, degradable material 214 may be configured to cover, close, etc. aperture 212, such that aperture release mechanism 210 may have a closed state. If degradable material 214 interacts with a substance (e.g., in the body lumen of a patient), degradable material 214 may degrade, thereby transitioning aperture 212 from a closed state to an open state. In some examples, degradable material 214 may be configured to allow for simple diffusion of the agent from an area of high concentration (e.g., an interior of the capsule) to an area of lower concentration (e.g., into the body lumen of the patient) via aperture 212.

In some embodiments, capsule 207 may include more than one configuration of aperture release mechanism 210. For example, capsule 207 may have a first set of aperture release mechanisms 210 (which may be apertures/openings) including a first degradable material and a second set of aperture release mechanisms 210 including a second degradable material. Any suitable number of configurations of aperture release mechanisms 210 may be present in capsule 207. In some embodiments, the number of configurations of aperture release mechanisms 210 may be associated with the number of internal sub-housings 222 of capsule 207. For example, a first interior sub-housing 222 may be associated with a first configuration of aperture release mechanisms 210, a second interior sub-housing 222 may be associated with a second configuration of aperture release mechanisms 210, a third interior sub-housing 222 may be associated with a third configuration of aperture release mechanisms 210, a fourth interior sub-housing 222 may be associated with a fourth configuration of aperture release mechanisms 210, etc. In another example, a first configuration of aperture release mechanisms 210 may activate in the acidic environment or the stomach and/or duodenum, releasing a first payload (e.g., agent) of a first interior sub-housing 222, and a second configuration of aperture release mechanisms 210 may activate in the basic environment or the jejunum and/or ileum, releasing a second payload (e.g., agent) of a second interior sub-housing 222.

In other examples, aperture release mechanisms 210 may be covered by a mechanical cover and may be operated by a mechanism similar to interaction point release mechanism 132. For example, aperture release mechanism 210 may include a cover that may be released by a latch or other similar type of mechanism in order to selectively deliver an agent (e.g., agent 10).

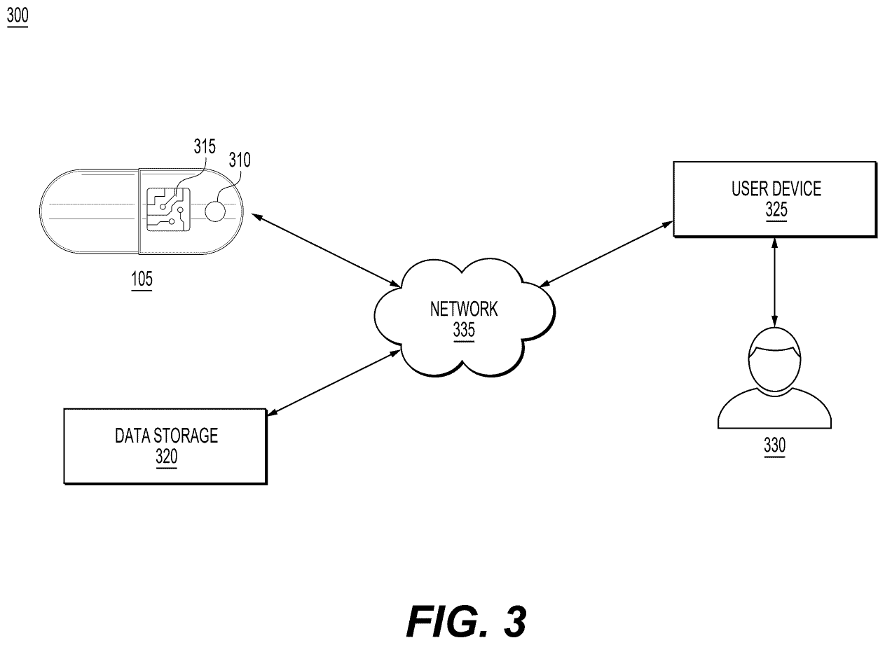

As mentioned above, medical device 105 may further include camera 310 or other imaging device and sensor 315. Although medical device 205 is not depicted with camera 310 or sensor 315, it will be appreciated that medical device 205 may include such elements. In some examples of medical device 105, camera 310 and/or sensor 315 may be omitted. Camera 310 and/or sensor 315 may be configured to communicate data collected by camera 310 and/or sensor 315 to one or more devices, e.g., to provide location data, chemical data, visual data, etc., as depicted in FIG. 3. Exemplary system 300 of FIG. 3 depicts medical device 105 configured to communicate with a data storage 320 and a user device 325, e.g., across a network 335. User device 325 may by any suitable device, e.g., a smart phone, a computer, a tablet, etc.

Camera 310 may be configured to capture camera data, such as photographs, videos, fluorescent signatures (e.g., of blood), light absorption properties (e.g., of blood), etc. Camera 310 may be any suitable type of device for capturing still and/or moving images. Camera 310 may have an image sensor. The camera data captured by camera 310 may be transmitted to data storage 320 and/or user device 325. In some embodiments, as discussed in further detail below, a user 330 may interact with the camera data, e.g., via user device 325.

Sensor 315 may be configured to capture sensor data, such as the presence of a substance (e.g., blood in the body lumen of a patient). Sensor 315 may be any suitable type of sensor (e.g., substance sensor), such as a pH sensor, a blood sensor, etc. In some embodiments, sensor 315 may be an electronic, optical, biological, or chemical component configured to detect or otherwise react in the presence of a substance in a body lumen. The sensor data captured by sensor 315 may be transmitted to data storage 320 and/or user device 325. In some embodiments, as discussed in further detail below, a user 330 may interact with the sensor data, e.g., via user device 325.

Camera 310, sensor 315, and other aspects of medical devices 105, 205 may communicate with data storage 320, user device 325, etc. Camera 310 and/or sensor 315 may be configured to communicate camera data and/or sensor data, respectively, to user device 325 (e.g., to a graphical user interface (GUI) associated with user device 325)

In some embodiments, user 330 may interact with camera data and/or sensor data via the GUI associated with user device 325, and/or user 330 may control the release mechanism associated with medical devices 105, 205. For example, user 330 may instruct (e.g., via the GUI associated with user device 325) release mechanism 132 of medical device 105 to activate.

In some embodiments, sensor 315 may be configured to determine and/or communicate (e.g., to a processor associated with user device 325, a GUI associated with user device 325, etc.) the location of the medical device (e.g., medical device 105), the location of an injury, a release status, etc. Sensor 315 may be configured to determine the location of the injury and/or medical device 105, 205 based on at least one of the time of peristalsis, the rate of motion through the body lumen, the composition of the surrounding contents (e.g., acidic, basic, neutral), the approximate time elapsed since ingestion of the medical device, etc. For example, if camera 310 and/or sensor 315 detect an acidic environment then a basic environment, it may be understood that the medical device has moved from the duodenum to the jejunum. In another example, the time elapsed since ingestion of the medical device may be compared against a standard or patient-specific digestion pattern (e.g., if it typically takes 4 hours after ingestion for a bolus to reach a patient's jejunum, it can be estimated that approximately 4 hours after ingestion the medical device may be located approximately in the jejunum).

More than one mechanism may be used to determine the location of medical device 105, 205. For example, sensor 315 may be configured to measure environmental pH and communicate the measured pH to user device 325 (e.g., to the processor associated with user device 325), and camera 310 may be configured to collect images and/or video of the body lumen and communicate the images and/or video to user device 325 (e.g., to the processor associated with user device 325). The processor associated with user device 325 may determine that the medical device is in either the jejunum or ileum based on a measured environmental basicity, and may further determine that the medical device is in the ileum based on the content of the images and/or videos and/or the amount of time elapsed since ingestion of the medical device (e.g., as measured by the images and/or videos captured by camera 310).

The status of the medical device may be determined based on sensor 315 sensing a change in the chemical components of the surrounding environment. For example, after the medical device has released the payload, sensor 315 may detect the presence of the payload in the body lumen and transmit this detection to a system (e.g., to the processor associated with user device 325). The processor associated with user device 325 may determine that the payload has been released based on the detection.

The medical device described herein may be incorporated in various embodiments or combinations of embodiments. In any embodiment, the medical device may house an agent, e.g., a hemostatic agent, a marking agent (e.g., a dye), etc. in an interior of a capsule.

In a first embodiment, a medical device may be configured to have diagnostic capabilities. As discussed in more detail herein in relation to FIGS. 1 and 3, medical device 105 may be a capsule 107, connected via connection point 120, with camera 310 and/or sensor 315. Alternatively, as discussed above, capsule 107 may include one or more openings/orifices, through which a payload (e.g., a hemostatic agent) may be delivered. Medical device 105 may be configured to communicate data collected by camera 310 and/or sensor 315 to one or more devices, e.g., data storage 320, user device 325, etc. For example, medical device 105 may be configured to communicate the location of medical device 105 in the body lumen, e.g., upon sensor 315 transmitting the detection of a substance (e.g., blood) to the processor associated with user device 325. Further, medical device 105 may be configured to be controllable by the one or more devices. For example, medical device 105 may receive a communication from user device 325, e.g., to release or not release an agent, and medical device 105 may or may not activate a release mechanism to release the agent based on the communication.

Medical device 105 may be swallowed by a patient with known or suspected bleeding. Any of the devices disclosed herein may be utilized following a procedure (e.g., post-operatively) or any other time a bleed may be suspected or of concern. As medical device 105 moves through the patient's gastrointestinal tract by natural peristalsis, it may encounter a bleeding site. Sensor 315 may detect the presence of blood, which may cause capsule 107 to rupture, dissolve, or burst, according to any of the mechanisms discussed above. In some examples, capsule 107 may be pressurized. Capsule 107 may cause a payload of capsule 107 (e.g., stored in interior housing 140) to be applied broadly to an area surrounding capsule 107. The payload may be a hemostatic agent (including any of the agents discussed above).

In one example, capsule 107 may be a pillcam, and sensor 315 and/or camera 310 may have fluorescent reading capabilities. Hemoglobin may have a unique fluorescent signature, which may be detected by camera 310 and/or sensor 315 of capsule 107. For example, camera 310 may evaluate specific light absorption properties that are unique to hemoglobin. Camera 310 and/or sensor 315 may cause capsule 107 to automatically deploy the payload. Alternatively, camera 310, sensor 315, and/or a transmitter of capsule 107 in communication with camera 310 and/or sensor 315 may communicate with user device 325, as discussed above. User device 325 may automatically instruct capsule 107 to deploy the payload, or a user may manually utilize user device 325 in order to deliver the payload (e.g., via a spring-loaded canister).

Alternatively, sensor 315 and/or camera 310 may detect electrical, optical, biological, and/or chemical properties of blood or another substance in a body lumen. In further alternatives, the patient may be administered an oral or intravenous compound that may be carried in the blood. Bleeding in a body lumen may result in the presence of the compound in the body lumen. The compound may include, for example, a specific molecule, enzyme, or fluorophore. When the compound is detected (e.g., via camera 310 and/or sensor 315), capsule 107 may deliver the payload, as described above.

In a second embodiment, a medical device 205 may be configured to degrade (e.g., automatically degrade) in certain conditions, releasing a payload, such as an agent (e.g., any of the agents discussed above). Medical device 205 may be administered as described above for medical device 105. As discussed in more detail herein in relation to FIGS. 2A and 2B, medical device 205 may be a single-piece capsule 207. Capsule 207 may include at least one interior sub-housing 222 that may be defined by at least one interior wall 215 and capsule wall 220. Capsule 207 may include any suitable number of sub-housings 222, e.g., one, two, three, four, etc. sub-housings 222. Each of the at least one interior sub-housing 222 may house at least one payload (e.g., hemostatic or other agent). For example, a first interior sub-housing 222 may house a first treatment agent, a second interior sub-housing 222 may house a second treatment agent, a third interior sub-housing 222 may house a third treatment agent, a fourth interior sub-housing 222 may house a marking agent, a fifth interior sub-housing 222 may house another treatment agent, etc.

As discussed above, capsule 207 (or portions thereof) may be composed of certain materials configured to degrade, e.g., via one or more of degradation of capsule wall 220, aperture release mechanisms 210, etc., in the presence of a substance (e.g., blood). For example, as discussed above, medical device 205 may include capsule wall 220 composed of a degradable material (e.g., degradable material 214). When capsule wall 220 interacts with a substance (e.g., blood, an activating substance, etc.), capsule wall 220 may degrade, releasing the agent.

In another example, aperture release mechanisms 210 may include a plurality of perforations or apertures 212 including degradable material 214. Degradable material 214 may cause apertures 212 to be in a closed state, and degradable material 214 interacting with a substance (e.g., blood, an activating substance, etc.) and degrading may cause apertures 212 to be in an open state. In alternatives, apertures 212 may be open (e.g., be in communication with interior housing 240) and not include degradable material 214. In some embodiments, capsule 207 may include more than one configuration of aperture release mechanism 210. For example, capsule 207 may have a first set of aperture release mechanisms 210 including a first degradable material and a second set of aperture release mechanisms 210 including a second degradable material. Any suitable number of configurations of aperture release mechanisms 210 may be present in capsule 207. In some embodiments, the number of configurations of aperture release mechanisms 210 may be associated with the number of internal housings 222 of capsule 207. For example, a first interior sub-housing 222 may be associated with a first configuration of aperture release mechanisms 210, a second interior sub-housing 222 may be associated with a second configuration of aperture release mechanisms 210, a third interior sub-housing 222 may be associated with a third configuration of aperture release mechanisms 210, a fourth interior sub-housing 222 may be associated with a fourth configuration of aperture release mechanisms 210, etc. In another example, a first configuration of aperture release mechanisms 210 may activate in the acidic environment or the stomach and/or duodenum, releasing a first payload (e.g., agent) of a first interior sub-housing 222, and a second configuration of aperture release mechanisms 210 may activate in the basic environment or the jejunum and/or ileum, releasing a second payload (e.g., agent) of a second interior sub-housing 222.

In some embodiments, capsule wall 125, 220 or portions thereof and/or aperture release mechanisms 210 may be composed of certain materials configured to degrade in the presence of an activating substance. For example, the material may degrade in the presence of an activating substance, but remain intact in the presence of other substances. The patient may have received an intravenous injection and/or oral formula of the activating substance prior to deployment of the medical device.

In a third embodiment, a medical device 105, 205 may be configured to release a marking agent, e.g., a dye. The medical device 105, 205 may be configured as described in the first embodiment and/or the second embodiment, but the agent housed within capsule 107, 207 may be a marking agent (e.g., a dye), such that when released, the marking agent may mark, dye, etc. the lesion. Exemplary marking agents are discussed above and include, for example, methylene blue, patent blue, brilliant blue, or an endoscopic tattoo. As discussed in more detail below, the location of the damage may then be determined based on the marked lesion, e.g., via X-ray, CT scan, endoscopic imaging, etc. The third embodiment provides a mechanism for tagging an area for subsequent evaluation and/or treatment (e.g., by endoscopic or surgical approach).

It should be noted that while these embodiments are discussed separately for clarity, it should be understood that aspects of an embodiment may be incorporated into another. For example, the third embodiment may be both configured to release a marking agent and include diagnostic capabilities. In another example, the first embodiment may be configured to have diagnostic capabilities and also configured to degrade in certain conditions to release an agent.

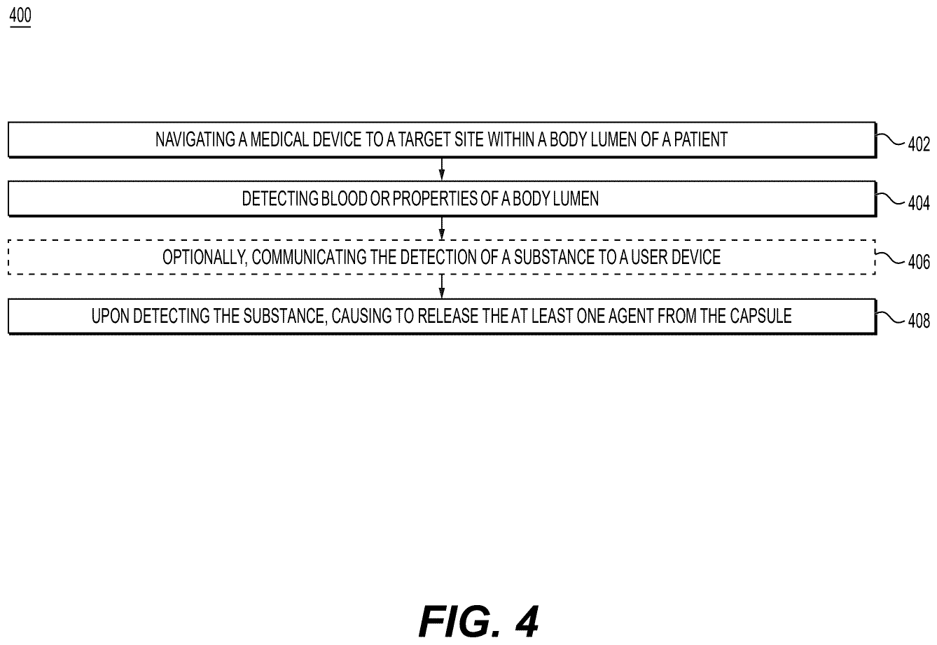

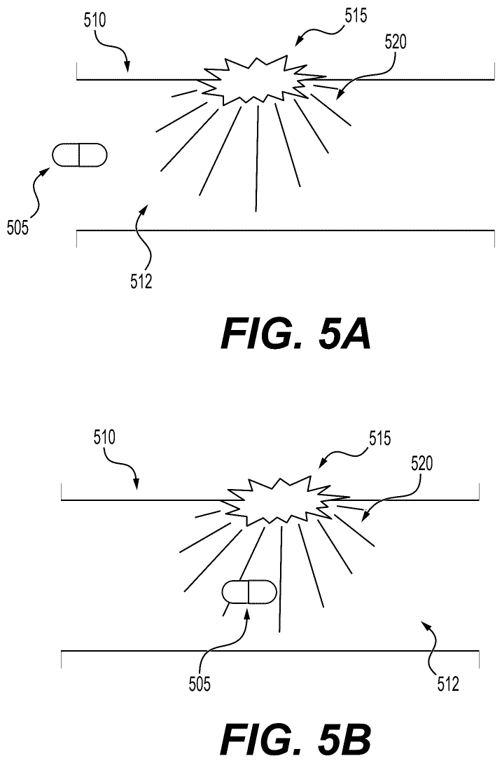

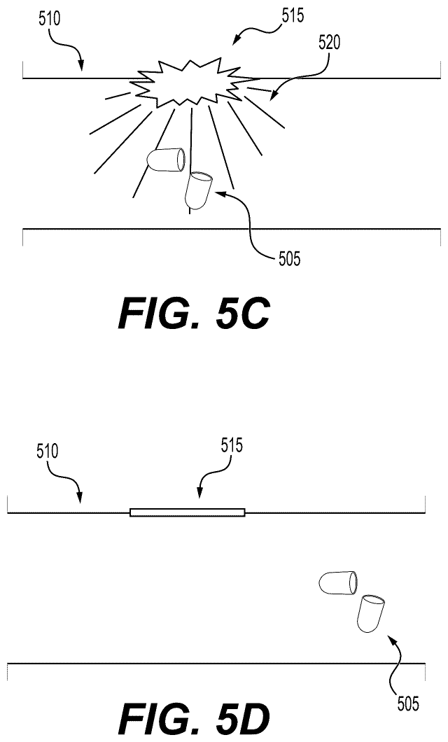

FIG. 4 illustrates a related method 400 of using an exemplary medical device, such as the medical devices of FIGS. 1-2B, e.g., medical device 105, medical device 205. FIGS. 5A-5D depict aspects of method 400. At step 402, a medical device may be navigated to a target site (e.g., an injury site) within a body lumen of a patient. In some examples, a patient may ingest the medical device (e.g., by swallowing the medical device), and peristalsis of the digestive tract may move the medical device through the patient's body lumen. In some examples, a duodenoscope or other type of scope may be utilized, e.g., to visualize the body lumen, to deliver medical device 105, 205, etc. As depicted in FIG. 5A, a medical device 505 (having any of the properties of medical devices 105, 205) in a closed and/or intact state (hereinafter “medical device”) may move through a body lumen 512 of a patient (e.g., the digestive tract, including the stomach, the duodenum, the jejunum, the ileum, etc.) toward the target site, such as an injury site. A luminal wall 510 may include an injury (or other type of lesion) 515. In some examples, injury 515 may be bleeding, such that a substance 520 (e.g., blood) is moving into body lumen 512.

At step 404, blood or properties of a body lumen (e.g., an acidic or basic environment) may be detected. As shown in FIG. 5B, as medical device 505 passes through body lumen 512, medical device 505 may come into contact with substance 520 (e.g., blood or properties of a body lumen), thereby activating the release mechanism associated with medical device 505. As discussed above, such release may be automatic or via user intervention.

The release mechanism associated with medical device 505 may be any release mechanism, such as degradation of capsule wall 125, 220, aperture release mechanisms 210, interaction point release mechanism 132, etc. For example, any of the above release mechanisms may be activated in the presence of a substance (e.g., blood, an activating substance, etc.). In another example, aperture release mechanisms 210 may be activated if degradable material 214 interacts with substance 520, causing degradable material 214 to degrade, as discussed herein. In another example, interaction point release mechanism 132 may be activated if at least one interaction point 135 interacts with substance 520, causing at least one interaction point 135 located on an exterior rim of capsule body 110 and at least one interaction point 135 located on the interior rim of capsule cap 115 to degrade and disconnect, as discussed herein.

As discussed herein, the type of substance that activates a release mechanism may vary. For example, if the substance is an activating substance, the patient may have received an intravenous injection and/or oral formula of the activating substance prior to initiation of step 402. Substance 520 (e.g., blood) may therefore include the activating substance, which may activate a release mechanism of medical device 505 configured to activate in the presence of the activating substance. In another example, a release mechanism may be configured to activate in the presence of blood and may activate upon encountering substance 520.

It should be noted that while medical device 505 is depicted interacting with substance 520 in relative proximity to injury 515, medical device 505 may interact with substance 520 in other areas of body lumen 512. For example, if injury 515 is located in a distal end of the jejunum, substance 520 may be detectable in the proximal end of the jejunum. Therefore, the release mechanism of medical device 505 may become activated prior to reaching injury 515. In another example, if substance 520 is an acidic environment, the release mechanism of medical device 505 may become activated in the stomach and/or duodenum, regardless of the location of injury 515.

In some examples, activation of the release mechanism may be controlled by user 330, e.g., via user device 325. For example, optionally, at step 406, the detection of substance 520 may be communicated to a user device, e.g., user device 325. For example, as substance 520 comes into contact with medical device 505, the detection of substance 520 (e.g., via sensor 315, camera 310, etc.) may be communicated to user device 325. In response to the communication, user 330 may input (e.g., via GUI associated with user device 325) an instruction (e.g., to activate, to not activate, etc.). The instruction may be communicated to the medical device, e.g., to camera 310, sensor 315, etc. In this way, activation of the release mechanism may be controlled, e.g., to prevent medical device 505 from prematurely releasing the agent. For example, user 330 may instruct a spring-loaded interaction point release mechanism 132 to release the agent at a point determined by user 330.

At step 408, upon detecting substance 520, the at least one agent may be released and/or caused to be released from the capsule, as depicted in FIG. 5C. It should be noted that while medical device 505 is depicted as having a connection point release mechanism, e.g., interaction point release mechanism 132, it should be noted that medical device 505 may include any suitable configuration of capsule, release mechanism, sensor, camera, etc., as discussed herein.

Based on the type of agent released, injury 515 may be dyed, marked, cauterized, mechanically treated, etc. For example, if the agent is a biocompatible dye, injury 515 may be marked, tagged, etc. with the biocompatible dye, which may enable injury 515 to be located, e.g., via X-ray, CT scan, MRI scan, endoscopy, etc. In another example, if the agent is a mechanical tamponade, the mechanical tamponade may expand in body lumen 512, putting mechanical pressure on injury 515. Mechanical tamponade may expand across the diameter and/or circumference of body lumen 512, such that pressure may be leveraged on injury 515 by mechanical tamponade pressing against the region of luminal wall 510 opposite injury 515. A mechanical tamponade may be configured to degrade or pass through body lumen 512 after any suitable amount of time (e.g., approximately one to thirty minutes). A mechanical tamponade may be configured to degrade or pass through body lumen 512 such that the gastrointestinal tract is not obstructed and/or the natural shedding schedule of the gastrointestinal tract is not hinder, impeded, etc. In a further example, the agent may be a molecule or compound suitable for activating a clotting cascade (e.g., kaolin clay, bentonite clay, etc.).

As depicted in FIG. 5D, after the at least one agent is released, medical device 505 may continue traveling in body lumen 512 and may be excreted by the patient. Alternatively, medical device 505 may entirely degrade within the body lumen.

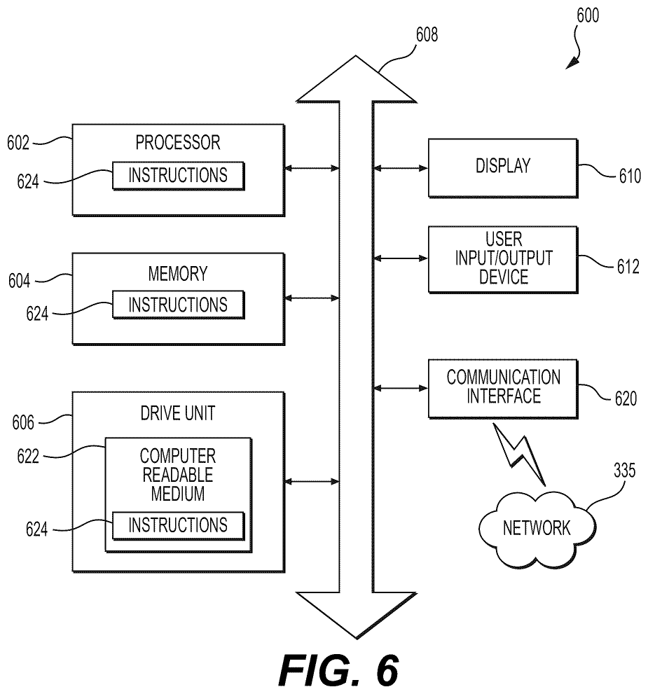

FIG. 6 depicts a simplified functional block diagram of a computer 600 that may be configured as a device for executing the methods disclosed here, according to exemplary embodiments of the present disclosure. For example, the computer 600 may be configured as a system according to exemplary embodiments of this disclosure. In various embodiments, any of the systems herein may be a computer 600 including, for example, a data communication interface 620 for packet data communication. The computer 600 also may include a central processing unit (CPU) 602, in the form of one or more processors, for executing program instructions. The computer 600 may include an internal communication bus 608, and a storage unit 606 (such as ROM, HDD, SDD, etc.) that may store data on a computer readable medium 622, although the computer 600 may receive programming and data via network communications. The computer 600 may also have a memory 604 (such as RAM) storing instructions 624 for executing techniques presented herein, although the instructions 624 may be stored temporarily or permanently within other modules of computer 600 (e.g., processor 602 and/or computer readable medium 622). The computer 600 also may include input and output ports 612 and/or a display 610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A medical device comprising:
a capsule wall defining at least one housing, wherein the at least one housing is configured to contain at least one agent; and
a sensor configured to detect a substance;
wherein the medical device is configured to release the at least one agent from the at least one housing upon interacting with the substance.

2. The medical device of claim 1, wherein the at least one agent includes a hemostatic agent or a dye.

3. The medical device of claim 1, wherein the sensor includes a camera, wherein the substance is blood, and wherein the camera is configured to detect blood.

4. The medical device of claim 3, wherein the camera is configured to detect a fluorescent signature of blood.

5. The medical device of claim 1, wherein the capsule wall includes an emulsion polymer configured to degrade in a basic solution.

6. The medical device of claim 1, wherein the at least one housing comprises more than one sub-housing.

7. The medical device of claim 1, further comprising a release mechanism, wherein the release mechanism includes a degradable material configured to degrade upon contact with the substance.

8. The medical device of claim 1, further comprising a pressurized release mechanism.

9. The medical device of claim 1, wherein the sensor includes an electronic component configured to communicate the detection of the substance to a user device.

10. The medical device of claim 1, wherein the substance includes one or more of an acidic environment, a basic environment, or an activating substance.

11. The medical device of claim 10, wherein the activating substance is administered intravenously or orally to a patient.

12. The medical device of claim 10, wherein the activating substance is at least one of an activating molecule, an enzyme, or a fluorophore.

13. The medical device of claim 1, wherein the capsule wall includes at least one aperture in a wall, the at least one aperture configured to transition from a closed state to an open state upon detection of the substance.

14. The medical device of claim 13, wherein each aperture of the at least one aperture includes a degradable material configured to degrade upon detection of the substance, such that the at least one aperture is transitioned from a closed state to an open state.

15. The medical device of claim 1, wherein at least a portion of the capsule wall includes a degradable material.

16. A medical device comprising:
a capsule wall defining at least one housing, wherein the at least one housing is configured to contain at least one agent; and
a sensor configured to detect blood, the sensor including an electronic component configured to communicate with a user device;
wherein the medical device is configured to deliver the at least one agent based on an instruction from the user device.

17. The medical device of claim 16, wherein the sensor is a camera configured to detect a fluorescent signature of blood.

18. A method, the method comprising:
administering a capsule to a patient, wherein the capsule contains at least one agent in an interior of the capsule; and
upon interaction with a substance in a body lumen of the patient, automatically releasing the at least one agent from the capsule.

19. The method of claim 18, wherein the capsule includes a wall, and wherein the at least one agent is automatically released by degradation of the wall upon interaction with the substance.

20. The method of claim 18, wherein the capsule includes a sensor configured to detect the substance, and wherein the at least one agent is automatically released when the sensor detects the substance.
